(19) United States
(12) Patent Application Publication
(10) Pub. No.: US 2026/0265677 A1
(43) Pub. Date: Sep. 10, 2026

(54) METHOD FOR PREPARING CULTURES OF LACTIC ACID BACTERIA, PRODUCTS AND CULTURE MEDIA THEREFORE

(71) Applicant: Chr. Hansen A/S, Hoersholm (DK)

(72) Inventor: Francesco Cristino FALCO, Hoersholm (IT)

(73) Assignee: Chr. Hansen A/S, Hoersholm (DK)

(21) Appl. No.: 18/873,526

(22) PCT Filed: Jun. 20, 2023
(86) PCT No.: PCT/EP2023/066647

§ 371 Date: Dec. 10, 2024

(30) Foreign Application Priority Data
Jun. 21, 2022 (EP) 22180169.9

(51) Int. Cl. C12N 1/20; C12N 1/02; C12N 1/04; C12N 1/38

(52) U.S. Cl. CPC C12N 1/20; C12N 1/02; C12N 1/04; C12N 1/38

(57) ABSTRACT

The present invention relates to microbial starter cultures. More specifically, a method for preparing a microbial culture such as a lactic acid bacteria (LAB) starter culture wherein at least one microbial strain such as a lactic acid bacteria and at least one non-protein-bound heme is inoculated in a culture medium.

### TECHNICAL FIELD

The present invention relates to the field of microbial starter cultures. More specifically, the invention provides a method for preparing a microbial starter culture under aeration. The microbial starter culture may be a lactic acid bacteria (LAB) starter culture wherein the lactic acid bacteria are inoculated in a culture medium and wherein the culture medium comprises at least one non-protein-bound heme. The novel method applies a vegetarian regulatory-compliant raw material. Therefore, the starter cultures obtained by the new method are useful in the manufacturing of vegetarian food-, feed- and pharmaceutical products.

### TECHNICAL BACKGROUND

Microbial cultures are used extensively in the food, feed and pharmaceutical industry in the manufacturing of fermented products including most dairy products such as cheese, yoghurt and butter, but also in meat, bakery, wine or vegetable products. Furthermore, microbial cultures are also used to produce proteins including enzymes and various kinds of useful compounds. Such microbial cultures are usually referred to as starter cultures and are produced at industrial propagation plants and distributed to the fermentation industry, such as to a dairy plant, where the starter culture is used in their production processes. In particular, cultures of lactic acid bacteria are widely used as starter cultures.

The production of lactic acid bacteria (LAB) starter cultures involves the inoculation of LAB cells in a specific fermentation medium with an appropriate number of the cells to be propagated under appropriate fermentation conditions. In the industrial setting much effort is put into obtaining a high concentration of propagated cells towards the end of the fermentation process. The fermentation conditions and the fermentation medium have to support growth of the cells in order to obtain the desired high biomass yields.

The methods currently applied for the production of starter cultures of lactic acid bacteria, such as Lactococcus lactis starter cultures, applies a non-vegetarian compliant source as a raw material in the fermentation media. The non-vegetarian compliant source is applied as an exogenous source. The exogenous source may be a heme source and it is added to support the respiratory process of the lactic acid bacteria. Due to the use of a non-vegetarian compliant heme source such starter cultures obtained by the known methods cannot be applied in vegetarian food-, feed and pharmaceutical products. Therefore, there is a need in the art to develop a respiratory process for the production of microbial starter cultures of e.g. lactic acid bacteria with yields similar to the processes known in the art and wherein the process applies a vegetarian compliant heme source.

Yeast cells have been implemented as vegetarian compliant heme source, as disclosed in WO2021/116311A1. However, yeast cells are cumbersome to produce, process and purify, which may lead to added cost. Yeast cells add dry matter besides the LAB biomass. Using yeast cell-based material in the form of yeast extract will add extra processing steps to the preparation of the non-protein-bound heme containing material.

Expression of heme using microorganisms can be done, as e.g. shown in EP3567109, entitled extracellular heme production method using metabolically engineered microorganism. However, it is unclear whether such heme is able to sustain cell cultures.

### SUMMARY OF THE INVENTION

A problem to be solved by the present invention is to provide a microbial culture such as a lactic acid bacterial culture applicable in the manufacturing of vegetarian food-, feed- and pharmaceutical products.

Accordingly, a first aspect the invention relates to a method for obtaining a microbial culture, said method comprises the steps of:
(i) culturing at least one microbial strain in a culture medium under aeration and obtaining a fermentate,
(ii) harvesting from the fermentate said at least one microbial strain to obtain the microbial culture,
   wherein the culture medium comprises at least one non-protein-bound heme.

In second aspect the invention relates to a culture obtainable by the method of the present invention.

In a third aspect the invention relates to a culture comprising at least one non-protein-bound heme.

A fourth aspect the invention relates to a culture medium comprising at least one non-protein-bound heme.

A fifth aspect of the invention relates to a method of preparing a food product, feed product, a pharmaceutical product, a dairy flavor and a cheese flavoring product, said method comprising adding an effective amount of the culture of the present invention to a food, feed or pharmaceutical product starting material and keeping the inoculated culture under conditions where the at least one microbial strain is metabolically active.

A sixth aspect of the invention relates to a fermented food, feed or pharmaceutical product obtainable by the method of the present invention.

A seventh aspect of the invention relates to the use of at least one non-protein-bound heme in a fermentation method and/or a fermentation process.

An eight aspect of the invention relates to a food product, feed product, a pharmaceutical product, a dairy flavor or a cheese flavoring product, comprising the culture according to the second or third aspect.

### DETAILED DISCLOSURE OF THE INVENTION

The inventors have developed a method for obtaining microbial cultures such as starter cultures of microbial strains (e.g. lactic acid bacteria), wherein non-protein-bound heme are used as a vegetarian compliant alternative heme source instead of a non-vegetarian compliant heme source. Applying a non-protein-bound heme as an exogenous heme source surprisingly showed to support respiration of microbial strains (such as lactic acid bacteria). The purified non-protein-bound heme is a vegetarian compliant raw material. The method provides yields comparable to the methods known in the art.

Prior to discussing the detailed embodiments of the invention a further definition of selected terms used herein is provided.

As used herein, the term “non-protein-bound heme” means free heme not associated to a protein that contains a heme prosthetic group.

As used herein, the term “fermentation” refers to a process of propagating or cultivating a microbial cell under aerobic or anaerobic conditions.

The term “starter culture” refers to a preparation comprising microbial cells that is intended for inoculating in a medium which is to be fermented.

In the present context, the term “yield” refers to the amount of biomass produced in a fermentation of a given volume. The yield may be measured in various ways; 1) As biomass per unit of volume measured (background subtracted) by the Optical Density at 600 nm (OD₆₀₀) of a 1 cm light path of the fermentation medium at the end of the fermentation; 2) by kg of F-DVS culture at the end of fermentation, by an “acidification activity” or acidification power of 4.8-5.2 according to the Pearce test; 3) by Packed Cell Volume (PCV) test, or; 4) cell count.

The term “F-DVS” refers to a so-called frozen Direct Vat Set cultures as described in the Examples.

The European legal framework on vegetarian claims is currently under revision and at present there are no harmonized rules. All claims under the European food legislation, vegan and vegetarian claims are any message or representation, which is not mandatory under European Union or national legislation, including pictorial, graphic or symbolic representation m in any form, which states, suggests or implies that a food has particular characteristics (Neli Sochirca (2018), EFFL, 6, page 514). Thus, in the present context the term “Vegetarian compliant heme source” refers to a heme source which is not obtained from or derived from an animal and/or multicellular organism. Contrary the term “non-vegetarian compliant heme source” refers to a heme source obtained from or derived from an animal and/or multicellular structure.

In an embodiment of the present invention the one or more microbial strain(s) is/are microbial strains not capable of respiratory growth without supplementation of components/substitute components of the respiratory chain. It will be appreciated that the supplementation of components/substitute components of the respiratory chain may be the supplementation of an exogenous heme source.

The at least one microbial strain may be selected from the group consisting of Lactococcus, Streptococcus, Lactobacillus now known as Ligilactobacillus, Holzapfelia, Amylolactobacillus, Bombilactobacillus, Companilactobacillus, Lapidilactobacillus, Agrilactobacillus,Schleiferilactobacillus, Loigolactobacilus, Lacticaseibacillus, Latilactobacillus, Dellagioa, Liquorilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus as described in Zheng et al, Int. J. Syst. Evol. Microbiol. DOI 10.1099/ijsem.0.004107, Leuconostoc., Oenococcus, Weissella, Pediococcus, Enterococcus, Bifidobacterium, Brevibacterium, Propionibacterium and combinations thereof. The majority of genera in this group are “lactic acid bacteria” however, an industrially important genus is Bifidobacterium, although phylogenetically unrelated, is sometimes included in the group of lactic acid bacteria since lactate is one of the main fermentation end products. The list also includes other industrially important starter cultures not included in the lactic acid bacteria genus belong to the genera Brevibacterium and Propionibacterium.

As used herein the term “lactic acid bacterium” (LAB) designates a gram-positive, microaerophilic or anaerobic bacterium which ferments sugars and produce acids including lactic acid (as the predominantly produced acid) andacetic acid. The industrially most useful lactic acid bacteria are found in the genera Lactococcus, Streptococcus., Lactobacillusnow known as Lactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus as described in Zheng et al, Int. J. Syst. Evol. Microbiol. DOI 10.1099/ijsem.0.004107, Leuconostoc., Oenococcus, Weissella, Pediococcus, and Enterococcus. As mentioned above another industrially important genus is Bifidobacterium, although phylogenetically unrelated, it is sometimes included in the group of lactic acid bacteria since lactate is one of the main fermentation end products.

Thus, in one embodiment the at least one microbial strain is a lactic acid bacteria, selected from the group consisting of Lactococcus, Streptococcus., Lactobacillus now known as Ligilactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus as described in Zheng et al, Int. J. Syst. Evol. Microbiol. DOI 10.1099/ijsem.0.004107, Leuconostoc., Oenococcus, Weissella, Pediococcus, Enterococcus, Bifidobacterium and combinations thereof.

Commonly used LAB starter culture strains of lactic acid bacteria are generally divided into mesophilic organisms having optimum growth temperatures at about 30° C. and thermophilic organisms having optimum growth temperatures in the range of about 40 to about 45° C.

It will be appreciated that the Lactobacillus genus taxonomy was updated in 2020. The new taxonomy is disclosed in Zheng et al. 2020 and the ones important to the present invention are summarized below:

| Current name | New name |
|---|---|
| Lactobacillus casei | Lacticaseibacillus casei |
| subsp. Casei | |
| Lactobacillus paracasei | Lacticaseibacillus paracasei subsp. paracasei |
| subsp. paracasei | Lacticaseibacillus paracasei subsp. tolerans |

Typical organisms belonging to the mesophilic group include Lactococcus lactis, Lactococcus lactis subsp. cremoris, Leuconostoc mesenteroides subsp. cremoris, Pediococcus pentosaceus, Lactococcus lactis subsp. lactis biovar. diacetylactis, Lactobacillus casei subsp. casei (Lacticaseibacillus casei) and Lactobacillus paracasei subsp. paracasei (Lacticaseibacillus paracasei subsp. paracasei and Lacticaseibacillus paracasei subsp. tolerans). Thermophilic lactic acid bacterial species include as examples Streptococcus thermophilus, Enterococcus faecium, Lactobacillus delbrueckii subsp. lactis, Lactobacillus helveticus, Lactobacillus delbrueckii sub sp. bulgaricus and Lactobacillus acidophilus.

Due to the fact that the amount and hence the concentration of the non-protein-bound heme, the lactic acid bacteria, the non-protein-bound heme or any other nutrients in the medium may change over time, e.g. due to incorporation into the microbial cells, it is necessary to refer to a specific point in time where the concentration of non-protein-bound heme has to be measured or determined. Therefore, the terms “initially” or “before fermentation” (also used herein interchangeably) when used in connection with the concentration of non-protein-bound heme, the lactic acid bacteria, the non-protein-bound heme or any other nutrients in the medium, refers to the concentration of non-protein-bound heme, the lactic acid bacteria, the non-protein-bound heme or any other nutrients present in the medium immediately before the microbial cells to be cultured are added to the medium.

For the overall fermentation process, it is however also possible to add non-protein-bound heme at any time prior to harvest. The addition of non-protein-bound heme can be done batch wise, or continuously. Thus, one important measure is the “total amount added” during the entire fermentation process.

A significant application of the starter culture according to the invention is as so-called probiotics. In the present context, the term “probiotic” is to be understood as microbial cultures which, when ingested in the form of viable cells by humans or animals, confer an improved health condition, e.g. by suppressing harmful microorganisms in the gastrointestinal tract, by enhancing the immune system or by contributing to the digestion of nutrients. A typical example of such a probiotically active product is “sweet acidophilus milk”.

The listing or discussion of an apparently prior-published document in this specification should not necessarily be taken as an acknowledgement that the document is part of the state of the art or is common general knowledge.

Embodiments, preferences and options for a given aspect, feature or parameter of the invention should, unless the context indicates otherwise, be regarded as having been disclosed in combination with any and all embodiments, preferences and options for all other aspects, embodiments, features and parameters of the invention. For example, embodiments relevant to the lactic acid bacteria culture obtainable by the method of the present invention may be equally applicable to the lactic acid bacteria starter culture. Also, embodiment stated in relation to the method of the present invention may be relevant to the products of the present invention and vice versa.

Embodiments of the present invention are described below, by way of examples only.

One aspect of the invention relates to a method for obtaining a microbial culture, said method comprises the steps of.
(i) culturing at least one microbial strain in a culture medium under aeration and obtaining a fermentate,
(ii) harvesting from the fermentate said microbial strain to obtain the microbial culture,
   wherein the culture medium comprises at least one non-protein-bound heme.

In an embodiment the invention relates to a method for obtaining a lactic acid bacteria culture, said method comprises the steps of:
(i) culturing at least one lactic acid bacteria culture in a culture medium under aeration and obtaining a fermentate,
(ii) harvesting from the fermentate said microbial strain to obtain the microbial culture,
   wherein the culture medium comprises at least one non-protein-bound heme.

In one embodiment the method of the present invention may further comprise a step of:
(iii) concentrating the microbial culture, to obtain a concentrated microbial culture.

In one embodiment the method of the present invention may further comprise a step of:
(iii) concentrating the lactic acid bacterial culture, to obtain concentrated lactic acid bacteria.

The concentrating may be performed using methods known in the art such as but not limited to centrifugation or ultra-filtration. In order to obtain an increased number of microbes (e.g. lactic acid bacteria) in the concentrate obtained in step (iii), it may be contemplated that the concentration factor in step (iv) is in the range from 2 to 20, such as in the range from 6-19, e.g. in the range from 7-18, such as 8-17, e.g. 9-16, such as 10-15, e.g. 11-14, such as 12-13, e.g. 2-4, such as 3-6.

Commercial starter cultures may commonly be distributed as frozen cultures. At the low temperatures at which such frozen cultures typically are maintained most metabolic activities in the cell ceases and cells can be maintained in this suspended, but viable, state for extended periods.

Concentrated frozen cultures are commercially very interesting since such cultures can be inoculated directly into the production container. By using such concentrated frozen cultures, the end-user avoids the otherwise obligatory, time-consuming intermediary fermentation step during which the starter culture are amplified, and the end-user furthermore reduces the risk of contamination drastically. Such concentrated cultures may be referred to as DVS—direct vat set™ cultures.

As an alternative to the concentrated frozen cultures concentrated freeze dried direct vat set™ cultures, FD-DVS™, may be prepared. Such cultures have the additional advantage that they can be shipped without refrigeration.

Thus, in an embodiment the method of the present invention may further comprise a step of:
(iv) freezing said microbial bacterial culture of step (ii) or the concentrated microbial culture in step (iii) to obtain a frozen microbial culture.

Thus, in an embodiment the method of the present invention may further comprise a step of:
(iv) freezing said lactic acid bacterial culture of step (ii) or the concentrated lactic acid bacteria in step (iii) to obtain a frozen lactic acid bacterial culture.

In order to remove liquid from the frozen microbial bacterial culture, the method of the present invention may further comprise a step of:
(v) sublimating water from said frozen microbial culture to obtain a dried microbial culture.

In order to remove liquid from the frozen lactic acid bacterial culture, the method of the present invention may further comprise a step of:
(v) sublimating water from said frozen lactic acid bacterial culture to obtain a dried lactic acid bacterial culture.

Step (v) may be carried out by a technique selected form the group consisting of spray drying, spray freezing, vacuum drying, air drying, freeze drying, tray drying and vacuum tray drying. In a further embodiment the method of the present invention further comprises a step of:
(vii) packing said frozen microbial culture obtained in step (iv) or the freeze-dried microbial culture obtained in step (v).

It may be appreciated that the method of the present invention further comprises a step of:
(vii) packing said frozen lactic acid bacterial culture obtained in step (iv) or the dried lactic acid bacterial culture obtained in step (v).

Often damaging effects of freezing and thawing on the viability of living cells has been observed. In general they are ascribed to cell dehydration and the formation of ice crystals in the cytosol during freezing.

However, a number of cryoprotective agents have been found to ensure that freezing occur in a controlled and minimally injurious manner, e.g. by ensuring that ice crystallization in the cytosol is precluded or minimized during freezing.

Preferably, at least one cryoprotectant is added to the harvested microbial culture or to the harvested lactic acid bacteria culture obtained in step (ii) or to the concentrated microbial culture or the concentrated lactic acid bacterial culture obtained in step (iii) Preferably, the cryoprotective agent(s) is selected from the group consisting one or more compound(s) involved in the biosynthesis of nucleic acids or one or more derivative(s) of any such compounds. Examples of preferred cryoprotective agent(s) suitable to be added to the harvested microorganism corresponds essentially to the preferred non-protein-bound heme(s) as described herein. Addition of such cryoprotective agent(s) to harvested microorganism is described in an earlier filed patent application with application number PCT/DK2004/000477. Preferred cryoprotective agent(s) described in PCT/DK2004/000477 are also preferred cryoprotective agent(s) of the present invention. The complete description of PCT/DK2004/000477 is incorporated by reference herein. In a further preferred embodiment of the invention the one or more cryoprotective agent(s) is/are selected from the group of nucleoside monophosphates. In a preferred embodiment at least one or the only cryoprotective agent is IMP. Carbohydrate or proteinaous type cryoprotectant agents are not in general described to increase the metabolic activity of thawed or reconstituted cultures. The cryoprotective agents of the invention may in addition to their cryoprotective activity also confers an increased metabolic activity (booster effect) of the culture when it is inoculated into the medium to be fermented, processed or converted. Thus one embodiment of the invention is a frozen or dried culture, wherein the cryoprotective agent is an agent or mixture of agents, which in addition to its cryoprotectivity has a booster effect. The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or dried (e.g. freeze dried) cultures may retain their viability, although they may have lost a significant portion of their metabolic activity e.g. cultures may lose their acid-producing (acidification) activity when kept stored even for shorter periods of time. Thus viability and booster effect has to be evaluated by different assays. Whereas viability is assessed by viability assays such as the determination of colony forming units, booster effect is assessed by quantifying the relevant metabolic activity of the thawed or reconstituted culture relative to the viability of the culture.

The acidifying activity assay described below is one example of an assay quantifying the relevant metabolic activity of the thawed or reconstituted culture.

Although the acid-producing activity is exemplified herein, this invention is intended to encompass the stabilization of any types of metabolic activities of a culture. Thus, the term “metabolic activity” refers to the oxygen removal activity of the cultures, its acid-producing activity, i. e. the production of e. g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In an embodiment of the invention the frozen culture contains or comprises from 0.2% to 20% of the cryoprotective agent or mixture of agents measured as % w/w of the frozen material. It is, however, preferable to add the cryoprotective agent or mixture of agents at an amount which is in the range from 0.2% to 15%, more preferably within the range of 0.2% to 10%, more preferably within the range of 0.5% to 7%, and more preferably within the range of 1% to 6% by weight, including within the range of 2% to 5% of the cryoprotective agent or mixture of agents measured as % w/w of the frozen material by weight. In a preferred embodiment the culture comprises approximately 3% of the cryoprotective agent or mixture of agents measured as % w/w of the frozen material by weight. The preferred amount of approximately 3% of the cryoprotective agent corresponds to concentrations in the 100 mM range. It should be recognized that for each aspect of embodiment of the invention the ranges may be increments of the described ranges.

In the case that the culture is a dried culture (e.g. freeze dried) it is preferred to add the cryoprotective agent or mixture of agents at an amount, which is in the range of 0.8% to 60% by weight, or within the range of 0.8% to 55% by weight, or within the range of 1.3% to 40% by weight, or within the range of 3% to 30% by weight, or within the range of 6% to 25% by weight, including the range of 10% to 24% by weight of the dried culture. In a preferred embodiment the dried culture (e.g. freeze dried) comprises approximately 16% of the cryoprotective agent or mixture of agents measured as % w/w of the dried culture.

Additionally, the frozen or dried culture may contain further conventional additives including nutrients such as yeast extract, sugars, antioxidants, inert gases and vitamins etc. Also surfactants including Tween® compounds can be used as further additive to the culture according to the invention. Further examples of such conventional additives, which in addition may be added to the culture according to the invention, may be selected from proteins, protein hydrolysates and amino acids. Preferred suitable examples of these include the ones selected from the group consisting of Glutamic acid, Lysine, Na-glutamate, Na-caseinate, Malt extract, Skimmed milk powder, Whey powder, Yeast extract, Gluten, Collagen, Gelatin, Elastin, Keratin, and Albumins or mixtures thereof.

More preferably the conventional additives is a carbonhydrate. Suitable examples of these include the ones selected from the group consisting of Pentoses (eg. Ribose, Xylose), Hexoses (e.g. fructose, mannose, Sorbose), Disaccharides (eg. Sucrose, Trehalose, Melibiose, Lactulose), Oligo saccharides (e.g. Raffinose), Oligofrutoses (eg. Actilight, Fribroloses), Polysaccharides (e.g. Maltodextrins, Xanthan Gum, Pectin, Alginate, Microcrystalline cellulose, Dextran, PEG), and Sugar alcohols (Sorbitol, Manitol and Inositol).

It is presently preferred that the ratio (wt %/wt %) of the at least one cryoprotectant to the concentrated microbial culture or the concentrated lactic acid bacteria culture is within the range from 1:0.5 to 1:5, such as from 1:1 to 1:4 or from 1:1% to 1:3.

An alternative embodiment of the invention is the method of preparing a microbial culture in increased yields as described herein and which further comprise that the concentrated microbial culture or the concentrated lactic acid bacterial culture obtained in step (iii) is dried by freeze drying, tray drying, spray drying, spray freezing, vacuum drying, air drying or any drying process which is suitable for drying of bacterial cultures.

The at least one non-protein-bound heme may be present in the culture medium or added to the culture medium before the at least one microbial strain and/or the lactic acid bacteria is/are added to the medium or alternatively, the at least one non-protein-bound heme may be added immediately after the at least one microbial strain and/or the lactic acid bacteria have been added to the culture medium.

In one embodiment, the at least one non-protein-bound heme is a protein is expressed from a microorganism. In a preferred embodiment, the non-protein-bound heme is the heme expressed according to EP3567109, entitled extracellular heme production method using metabolically engineered microorganism.

In one embodiment the composition comprising the at least one non-protein-bound heme, has been inactivated. Several inactivation methods can be used to achieve the objective of inactivating native biological catalytical activity, such as pH (base) inactivation, enzymatic digestion, or heat inactivation. In a preferred embodiment, the inactivation is heat inactivation. The heat inactivation may be performed by any method known in the art, such as but not limited to autoclavation and/or UHT.

The inventors surprisingly found that adding of a heat stabilizing compound, enabled industrial processing of the non-protein-bound heme, while simultaneously enabling high growth yields. In one embodiment, the culture medium further comprises a heat stabilizing compound selected from the group consisting of: polyols, sugars, biopolymers, amino acids, salt, polymers and non-ionic detergents. The heat stabilizing compound may be selected from the group consisting of: Sorbitol, Glycerol, Propylene glycol, Mannitol, Xylitol, Propanediol, Trehalose, Sucrose, Lactose, Maltose, Glucose, Levan (fructose homopolysaccharide), Dextrans, Dextran sulfate, Gelatins (type A and B), Hydroxyethyl starch, poly-L-glutamic acid, poly-L-lysine, Fucoidan, Pentosan polysulfate, Keratan sulfate, poly-Aspartate, poly-Glutamate, Hydroxyethylcellulose, Hydroxypropyl-β-Cyclodextrin, Glycine, L-Arginine hydrochloride, arginine, Proline, Lysine, Histidine, Aspartic acid, Glutamic acid, Acetate, Citrate, Sodium chloride, Phosphates, Ascorbate, poly(acrylic acid) randomly modified with n-octylamine and isopropylamine (A8-35), Polyethylene Glycols (PEG), Polyvinyl sulfate, Polysorbate 20, Polysorbate 80, Triton X-100, Pluronic F68, Pluronic F88, Pluoronic F-127, Brij 35 (polyoxyethylene alkyl ether). In a preferred embodiment, the heat stabilizing compound is sorbitol.

In one embodiment, the non-protein-bound heme is a protein without native biological activity.

In one embodiment, the non-protein-bound heme is microbially produced. In one embodiment, the non-protein-bound heme is indirectly derived from, or directly produced by, an organism of the genus Aspergillus, such as Aspergillus niger. In one embodiment, the non-protein-bound heme is indirectly derived from, or directly produced by, an organism of the genus Pichia, such as Pichia pastoris. In one embodiment, the non-protein-bound heme is indirectly derived from, or directly produced by an organism of the genus Saccharomyces, such as Saccharomyces cerevisiae. In one embodiment, the non-protein-bound heme is indirectly derived from, or directly produced by an organism of the genus Escherichia, such as Escherichia coli. In one embodiment, the non-protein-bound heme is indirectly derived from, or directly produced by, an organism of the genus Bacillus, such as a non-sporulating Bacillus.

The at least one non-protein-bound heme is added to or present in the culture medium as a raw material intended to aid fermentation. The present inventors surprisingly discovered that the application of a non-vegetarian source in the culture medium may be replaced with at least one non-protein-bound heme without a decrease in yield.

An aspect of the present invention therefore relates to the use of at least one non-protein-bound heme in a fermentation method and/or fermentation process.

The culture medium may be a complex fermentation medium.

The complex fermentation medium may be any complex fermentation medium known in the art however the complex fermentation medium may comprise compounds selected from the group consisting of lactose, nutrients, vitamins tryptone, soya peptone, yeast extract, Ascorbic acid, Magnesium sulphate, milk and combinations thereof.

In one embodiment, the non-protein-bound heme is added at a level allowing respiration above the natural level of oxygen consumption the cells would be able to support. The non-protein-bound heme stimulates aerobic microbial growth in a dose-dependent manner such that oxygen consumption, as a measure of microbial growth, peaks earlier and at a faster rate in comparison to a cultivation without said non-protein-bound heme Thus, in one embodiment, the oxygen consumption in the fermentate reaches its maximum value in less than 12 hours, such as less than 10 hours or less than 8 hours.

In one embodiment, the oxygen consumption in the fermentate reaches 0.04 mol 02/L/h in less than 10 hours or less than 8 hours.

Oxygen consumption can be measured using any method know to a person skilled in the art.

In one embodiment the culture medium in step (i) comprises at least 0.005% w/w of the at least one non-protein-bound heme before fermentation (i.e. before the at least one microbial strain(s) is/are added), such as 0.008% w/w, 0.01% w/w, 0.014% w/w, 0.03% w/w, or 0.5% w/w, such as in the range from 0.008% w/w to 0.014% w/w, 0.01% w/w to 0.03% w/w, 0.01% w/w to 0.5% w/w, or 0.005% w/w to 0.5% w/w of the at least one non-protein-bound heme to the weight of the culture medium (i.e. before the at least one microbial strain(s) is/are added).

In a preferred embodiment, the concentration of non-protein-bound heme in the culture medium in step (i) is between 0.008% w/w and 0.014% w/w.

A concentration of non-protein-bound heme in culture medium of about 0.008% w/w, such as 0.008% w/w, corresponds to about 9-10 ppm heme and is particularly suitable for UHT sterilization.

A concentration of non-protein-bound heme in culture medium of about 0.014% w/w, such as 0.014% w/w, corresponds to about 16-17 ppm heme and is particularly suitable for filter sterilization.

In a further embodiment the culture medium in step (i) comprises at least 0.5% w/w of the microbial inoculation culture such as an lactic acid bacteria inoculation culture before fermentation, such as at least 1% w/w, e.g. 1.5% w/w, such as 2% w/w, e.g. 2.5% w/w, such as 3% w/w, e.g. 3.5% w/w, such as 4% w/w, such as in the range from 0.5-4% w/w, e.g. 1-3.5% w/w, such as 1.5-3% w/w, e.g. 2-2.5% w/w of the at lactic acid bacteria inoculation culture before fermentation to the weight of the culture medium (i.e. before the at least one microbial strain(s) is/are added). The inoculation culture may be made according to the method specified in Example 1.

In one embodiment, the non-protein-bound heme is added to a concentration of between about 0.1 g/kg fermentate and about 10 g/kg fermentate.

Surprisingly, by the method of the present invention it is occasionally possible to obtain a microbial culture such as lactic acid bacteria cultures that are sufficiently concentrated to be used for production of F-DVS without concentration of the culture. However even when the present method applied most cultures need to be concentrated to obtain starter cultures of commercial interest. Such cultures may preferably be harvested and concentrated by centrifugation or ultra-filtration.

Further, a preferred embodiment is wherein the culturing is performed in a large-scale fermentor comprising of from 5 L to 100.000 L culture medium, preferably of from 300 L to 20.000 L culture medium.

A preferred embodiment is wherein the culturing comprising control of temperature and/or pH.

In an embodiment the culture medium in step (i) and/or step (ii) comprises one or more microbial strain(s) is/are microbial strains that are not capable of respiratory growth without supplementation of components/substitute components of the respiratory chain.

In an embodiment the culture medium in step (i) and/or step (ii) comprises at least one microbial strain selected from the group consisting of Lactococcus, Streptococcus., Lactobacillusnow known as Lactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus as described in Zheng et al, Int. J. Syst. Evol. Microbiol. DOI 10.1099/ijsem.0.004107, Leuconostoc., Oenococcus, Weissella, Pediococcus, Enterococcus, Bifidobacterium, Brevibacterium, Propionibacterium and combinations thereof.

In an embodiment the culture medium in step (i) and/or step (ii) comprises at least one lactic acid bacteria selected from the group consisting of Lactococcus, Streptococcus., Lactobacillusnow known as Lactobacillus, Holzapfelia, Amylolactobacillus, Bombilactobacillus, Companilactobacillus, Lapidilactobacillus, Agrilactobacillus,Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus as described in Zheng et al, Int. J. Syst. Evol. Microbiol. DOI 10.1099/ijsem.0.004107, Leuconostoc., Oenococcus, Weissella, Pediococcus, Enterococcus. and Bifidobacterium.

In an embodiment the culture medium in step (i) and/or step (ii) comprises one or more mesophilic organisms selected from the group comprising Lactococcus lactis, Lactococcus lactis subsp. cremoris, Leuconostoc mesenteroides subsp. cremoris, Pediococcus pentosaceus, Lactococcus lactis subsp. lactis biovar. diacetylactis, Lactobacillus casei subsp. casei (new name Lacticaseibacillus casei), Lactobacillus paracasei subsp. Paracasei ((Lacticaseibacillus paracasei subsp. paracasei and Lacticaseibacillus paracasei subsp. tolerans).) and Oenococcus oeni.

In a further embodiment the culture medium in step (i) and/or step (ii) comprises one or more thermophilic organisms having optimum growth temperatures at about 40° C. to about 45° C.

In yet and embodiment the culture medium in step (i) and/or step (ii) comprises one or more thermophilic organisms selected from the group comprising Streptococcus thermophilus, Enterococcus faecium, Lactobacillus delbrueckii subsp. lactis, Lactobacillus helveticus, Lactobacillus delbrueckii subsp. bulgaricus and Lactobacillus acidophilus.

In an embodiment, the culture medium in step (i) and/or step (ii) is a LD-culture that comprises one or more organisms selected from the group comprising Lactococcus lactis subsp. lactis, Lactococcus lactis subsp. cremoris, Lactococcus lactis subsp. lactis biovar. diacetylactis and Leuconostoc mesenteroides subsp. cremoris. In the present context the term “LD-culture” is to be understood as the combination of the species Lactococcus lactis and the species Leuconostoc.

It may be appreciated that the culture medium in step (i) and/or step (ii) is an O-culture that comprises one or more organisms selected from the group comprising Lactococcus lactis subsp. lactis and Lactococcus lactis subsp. cremoris. In the present context “O-culture” is to be understood as a culture medium comprising Lactococcus lactis subsp. lactis and Lactococcus lactis subsp. cremoris. 0-cultures are typically used to make cheese without holes (Cheddar, Chesh-ire, Feta). The particular culture is commercially available under the name R 604 from Chr. Hansen A/S, Denmark (catalogue no. 200113).

In a preferred embodiment culture medium in step (i) and/or step (ii) is a culture comprising Lactococcus lactis.

In order to obtain maximum yield, it may be preferred that the harvest in step (ii) is performed between 5 and 24 hours after the start of the culture.

The method of the present invention may further comprise storage of the harvested microbial culture or the lactic acid bacteria culture obtained in step (ii) or the concentrated microbial culture or the lactic acid bacteria culture obtained in step (iii).

Due to the high yield of the method the microbial culture in the fermentate obtained in step (i) may comprises in the range of 2.0E+10-5.0E+10 active microbial cells/g microbial culture, such as 2.5E+10-4.5E+10, e.g. 3.0E+10-4.0E+10 active microbial cells cells/g microbial culture. Likewise, the microbial culture in the fermentate obtained in step (i) may comprise in the range of 2,0E+10-5,0E+10 total microbial cells/g microbial culture, such as 2.5E+10-4.5E+10, e.g. 3.0E+10-4.0E+10 total microbial cells/g microbial culture. In table 2 in the experimental part it can be seen that the number of active lactic acid bacterial cells and the total number of lactic acid bacterial cells are almost identical thus, indicating that the lactic acid bacterial culture and the lactic acid bacterial starter culture obtainable by the present invention has high viability.

Due to the high yield of the method the lactic acid bacterial culture in the fermentate obtained in step (i) may comprises in the range of 2.0E+10-5.0E+11 active lactic acid bacterial cells/g lactic acid bacterial culture, such as 2.5E+10-4.5E+10, e.g. 3.0E+10-4.0E+10 active lactic acid bacterial cells/g lactic acid bacterial culture. Likewise, the lactic acid bacterial culture in the fermentate obtained in step (i) may comprise in the range of 2,0E+10-5,0E+10 total lactic acid bacterial cells/g acid bacterial culture, such as 2.5E+10-4.5E+10, e.g. 3.0E+10- 4.0E+10 total lactic acid bacterial cells/g acid bacterial culture. In table 2 in the experimental part it can be seen that the number of active lactic acid bacterial cells and the total number of lactic acid bacterial cells are almost identical thus, indicating that the lactic acid bacterial culture and the lactic acid bacterial starter culture obtainable by the present invention has high viability.

The number of active and/or total cells are determined using flowcytometry which is technique known to the skilled person.

In a preferred embodiment, wherein said increased yield of the harvested microbial strain(s) e.g. lactic acid bacteria or the microbial culture such as a lactic acid bacterial culture of the method is increased by a factor of at least 1.2, preferably by a factor of at least 1.3, more preferably by a factor of at least 1.4, even more preferably by a factor of at least 1.5 and most preferably by a factor of at least 1.6 compared to the Anaerobic process excluding heme source process.

In a second aspect the invention relates to a microbial culture, such as a starter culture, obtainable by the method of the first aspect of the invention. The microbial culture, such as the starter culture, may be provided as a culture concentrate, such as a starter culture concentrate.

In third aspect the invention relates to a microbial culture such as a starter culture comprising at least one non-protein-bound heme.

A fourth aspect relates to a culture medium comprising at least one non-protein-bound heme.

A fifth aspect the invention relates to a method of preparing food product, feed product, a pharmaceutical product, a dairy flavor and a cheese flavoring product, said method comprising adding an effective amount of the culture according to the second or third aspect, to a food, feed or pharmaceutical product starting material and keeping the inoculated culture under conditions where the at least one microbial strain is metabolically active.

Preferably the food product of the fifth aspect of the invention is selected from the group consisting of a milk-based product, a vegetable product, a meat product, a beverage, a fruit juice, a wine, a bakery product, a dairy flavor and a cheese flavoring product.

Preferably the milk-based product is selected from the group consisting of a cheese, yoghurt, a butter, an inoculated sweet milk and a liquid fermented milk product.

In a sixth aspect the invention relates to a fermented food, feed or pharmaceutical product obtainable by the method of first aspect.

A seventh aspect of the invention relates to the use of at least one non-protein-bound heme in a fermentation method and/or a fermentation process.

An eight aspect relates to food product, feed product, a pharmaceutical product, a dairy flavor or a cheese flavoring product, comprising the culture according to the second or third aspect.

The invention is further illustrated in the following non-limiting examples and the figures.

### FIGURES

FIG. 1 is a graph showing Dissolved Oxygen (DO) profiles for aerated bacteria grown in medium without pH adjustment and with addition of heme supplement according to an embodiment.

FIG. 2 is a graph showing Dissolved Oxygen (DO) profiles for aerated bacteria grown in medium with pH adjustment and with addition of heme supplement according to an embodiment.

FIG. 3 are graphs showing Dissolved Oxygen (DO) profiles for aerated bacteria grown in medium without pH adjustment and with addition of heme supplement according to an embodiment.

FIG. 4 are graphs showing Dissolved Oxygen (DO) profiles for aerated bacteria grown in medium with pH adjustment and with addition of heme supplement according to an embodiment.

FIG. 5 are graphs showing Dissolved Oxygen (DO) profiles for aerated bacteria grown in medium with addition of heme supplement according to an embodiment, where the supplement has been sterile filtered (FIG. 5A) or UHT treated (FIG. 5B).

FIG. 6 are graphs showing Dissolved Oxygen (DO) profiles for aerated bacteria grown in medium with addition of heme supplement according to an embodiment, where the supplement has been sterile filtered (FIG. 6A) or UHT treated (FIG. 6B).

FIG. 7 is a graph showing Dissolved Oxygen (DO) profiles for aerated bacteria grown in medium with addition of heme supplement according to an embodiment.

FIG. 8 are graphs showing Carbon dioxide Evolution Rate (CER) for aerated bacteria grown in medium with addition of heme supplement according to an embodiment, where the supplement has been sterile filtered (FIG. 8A) or UHT treated (FIG. 8B).

FIG. 9 are graphs showing Oxygen Uptake Rate (OUR) for aerated bacteria grown in medium with addition of heme supplement according to an embodiment, where the supplement has been sterile filtered (FIG. 9A) or UHT treated (FIG. 9B).

FIG. 10 is a picture of samples taken during Packed Cell Volume (PCV) % measurements for End of Fermentation (EoF) culture broth samples from aerated bacteria grown in medium with addition of heme supplement according to an embodiment, where the supplement has been sterile filtered (FIG. 10A) or UHT treated (FIG. 10B).

FIG. 11 are bar graphs showing active cell count measurements (FIG. 11A) and acidification activities (FIG. 111B) measured for End of Fermentation (EoF) cold culture broth samples as a function of the supplemented heme levels.

### EXAMPLES

### Strains

The examples involve strains listed in Table 1. All strains have been deposited at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany. The accession number given in Table 1.

The applicant requests that a sample of the deposited microorganisms stated below may only be made available to an expert, subject to available provisions governed by Industrial Property Offices of States Party to the Budapest Treaty, until the date on which the patent is granted.

TABLE 1
Overview of strains used in examples

| Strain | Accession No. | Deposit date |
|---|---|---|
| Lactococcus lactis | DSM 24648 | 2011 Mar. 15 |

### Media Preparation

In the examples, bacterial growth media including non-protein-bound heme is used. It is called non-protein bound heme because the heme is not associated to a protein that contains a heme prosthetic group. Such heme can be obtained through microbial expression, as described in EP3567109, Extracellular heme production method using metabolically engineered microorganism.

Heme expressed according to the method described in EP3567109 was obtained, either as a supernatant powder or as a biomass powder. The supernatant powder consists of dried supernatant powder containing secreted heme at an approximate concentration of 1-5% (w/w). The biomass powder consists of dried cellular material (again as a powder) containing non-secreted heme at an approx. conc of 5-10% (w/w). Stock solutions were prepared by dissolving the desired quantity of heme containing material from either the dry supernatant or biomass powder in 60 mM NaOH aqueous solution to obtain heme-rich stock solutions with a final pH usually in between 10 and 11. This was necessary, since neither of the two powders resulted to be soluble in water without increasing the pH level.

### Example 1: Fermentations in BioLector

Stock solutions of both supernatant powder and biomass powder were prepared. Lactococcus lactis cells (DSM 24648) were grown in standard medium, but with either non-protein-bound heme from supernatant powder or biomass powder added. BioLector microbioreactor from Beckman Coulter was used, according to standard instructions provided by the manufacturer.

FIG. 1 is a graph showing Dissolved Oxygen (DO) profiles for aerated cell cultivations in standard medium without pH adjustment (i.e., pH equal to 7.3) supplemented with sterile filtered heme from dry supernatant powder in the concentration range 0.500-0.010% w/w.

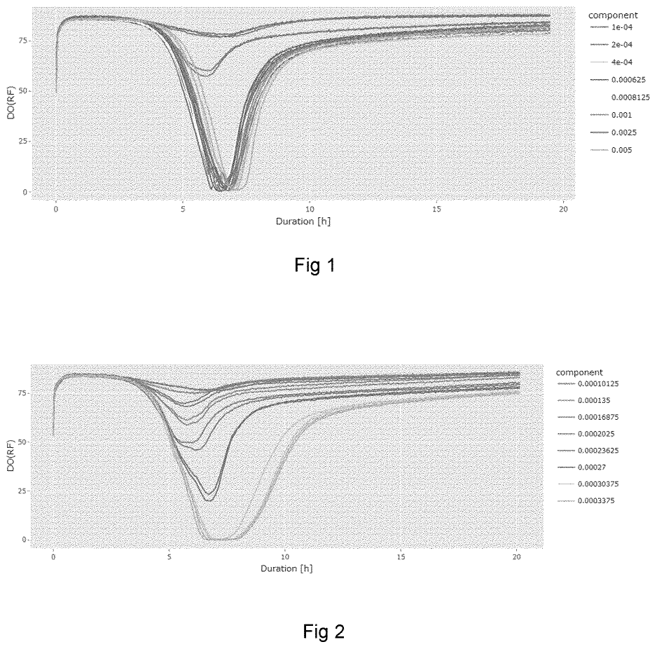

FIG. 2 is a graph showing Dissolved Oxygen (DO) profiles for aerated cell cultivations in standard medium with pH adjusted to 6.3 and then supplemented with sterile filtered Heme from dry supernatant powder in the concentration range 0.033-0.010% w/w.

This example shows that heme contained in the dry supernatant powder was not able to support heme-induced respiration under aerobic conditions in any of the investigated concentrations. No base consumption was observed for any of the conditions shown in Figure Error! Reference source not found. 1 and FIG. 2. This was noticed in connection to a premature arrest of the acidification of the cultivation medium presumably linked to an early growth arrest. Consequently, the pH set point of 6.2 was never reached during any of the aerated cultivation run with the Heme from dry supernatant powder.

However, the example shows that the heme contained in the dry biomass powder was capable of supporting heme-induced respiratory growth in an aerated culture. For high concentrations of the dry biomass powder stock solutions, an onset of respiration inhibition was observed.

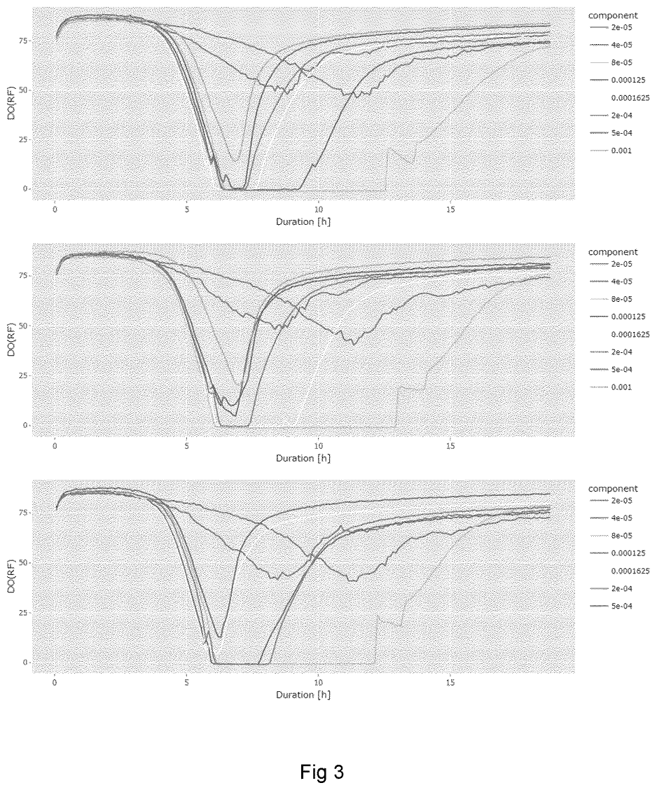

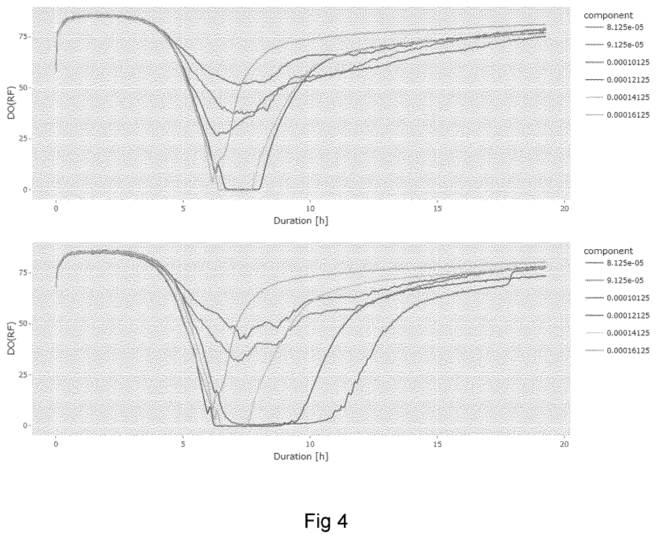

In particular, the two BioLector trials with the filter sterilized Heme biomass powder showed that respiratory metabolism occurred:
1) In the concentration range 0.020-0.008% w/w when the cell medium pH was not adjusted (i.e., with a pH around 7.3) as shown in FIG. 3.
2) In the concentration range 0.014-0.010% w/w when the cell medium pH was adjusted to 6.3 previous supplementation with sterile filtered Heme (resulting in a starting pH value in the interval 6.5-6.7) as presented in FIG. 4.

The onset of iron dependent heme cytotoxicity (Sawai, H., Yamanaka, M., Sugimoto, H., Shiro, Y., & Aono, S. (2012). Structural basis for the transcriptional regulation of heme homeostasis in Lactococcus lactis. Journal of Biological Chemistry, 287(36), 30755-30768.; Joubert, L., Derre-Bobillot, A., Gaudu, P., Gruss, A., & Lechardeur, D. (2014). HrtBA and menaquinones con-trol haem homeostasis in Lactococcus lactis. Molecular Microbiology, 93(4), 823-833.) in cells was observed for a concentration of Heme from dry biomass powder larger than 0.020% when medium pH was not adjusted (FIG. 3) or for a concentration larger than 0.014% when medium pH was adjusted to 6.3 previous heme supplementation (FIG. 4).

Hemin (olive-green when in aq. solution; Tahoun, M., Gee, C. T., McCoy, V. E., Sander, P. M., & Mûller, C. E. (2021). Chemistry of porphyrins in fossil plants and animals. RSC Advances, 11(13), 7552-7563) consists of a ferric [Fe(III)] complex of Protoporphyrin IX associated with a chloride ion which must be reduced to iron(II) in order to restore the ETC. Iron porphyrins should be relatively stable complexes, although the prolonged interaction of hemin with oxygen can possibly result into the breakage of the porphyrin ring, thereby removing the iron and its ability to react with cytochromes (Hogle, S. L., Barbeau, K. A., & Gledhill, M. (2014). Heme in the marine environment: From cells to the iron cycle. Metallomics, 6(6), 1107-1120). Protoporphyrin IX alone results in a red-brown aq. solution (Tahoun et al., 2021). It could be speculated that oxidation of the secreted heme contained in the dry supernatant powder could have possibly led to disruption of the hemin molecule, thus also affecting cell growth during aerobic cultivation of cells in standard medium supplemented with sterile filtered Heme from dry supernatant powder.

### Example 2: Fermentations 2 L Bioreactor

Stock solution of biomass powder was prepared. Lactococcus lactis cells (DSM 24648) were grown in standard medium, but with non-protein-bound heme from biomass powder added. Fermentation was conducted in 8×2 L fermenters in order to monitor online the evolution of the cultivation parameters of interest and harvest the obtained microbial mass to evaluate the performance of each culture in terms of PCV (%) and acidification activity.

The cultivations were run in 2 L Sartorius Biostat B bioreactors using a cascade control where both stirrer speed and air flow were regulated to maintain the DO around the setpoint value of 50%. The starting pH before inoculation was adjusted to 6.5 and then, following the acidification of the medium it was kept around a setpoint value of 6.2 through automatic base addition. The culture medium was supplemented with Heme from dry biomass powder either as a 0.1% (w/w) sterile filtered (FM15, FM16, FM17 and FM18) or a 0.1% (w/w) UHT treated (FM19, FM20, FM21 and FM22) stock solution. The tested concentration levels of heme (i.e., 0.008%, 0.010%, 0.012% and 0.014%) were identical for both sterilization treatment.

### Fermentation Conditions of the Cultures

The fermentation was performed in a 2 L Lab scale fermentation tank with aeration at 30° C. using 1% (w/w) of the culture mentioned above as inoculum and one of the abovementioned non-protein-bound heme as heme source. For aerobic fermentation as a positive control, the same conditions as for the aerobic fermentation was applied with aeration in a proprietary vegetarian friendly complex fermentation medium of Chr. Hansen A/S including a non-vegetarian heme source. For anaerobic fermentation as a negative control, the same conditions as for the aerobic fermentation was applied but without aeration in a proprietary vegetarian friendly complex fermentation medium of Chr. Hansen A/S excluding heme source.

The medium was sterilized either by filtration or by UHT-treatment (141° C. for 8-10 seconds).

The finished medium had a pH of 6.5.

The cultures were allowed to acidify to pH 6.0. The pH was subsequently maintained at 6.0 by controlled addition of 27% NH₄OH.

When no further base consumption was detected, the respective culture was cooled down to about 10° C.

Following cooling, the bacteria in the culture media were concentrated 6-18 times by centrifugation and subsequently frozen as pellets in liquid nitrogen at one atmosphere of pressure to produce a so-called frozen Direct Vat Set culture (F-DVS). The F-DVS pellets were stored at—50° C. until further analysis.

Lactococcus lactis changes metabolism profoundly when going from anaerobic to respiratory growth. Compared to anaerobic growth, biomass is approximately doubled, and acid production is reduced during respiratory growth. A key feature of respiratory growth is the reduction of dissolved oxygen (DO, measured in %)

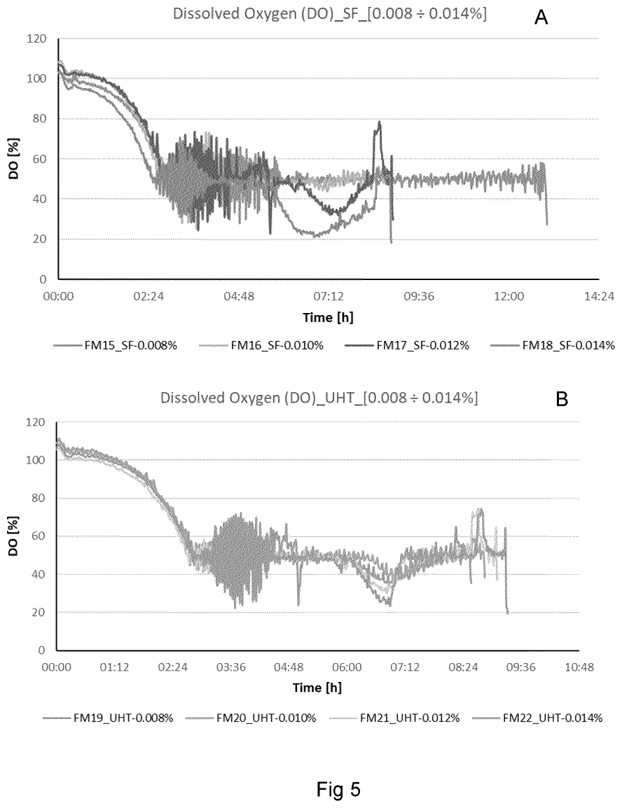

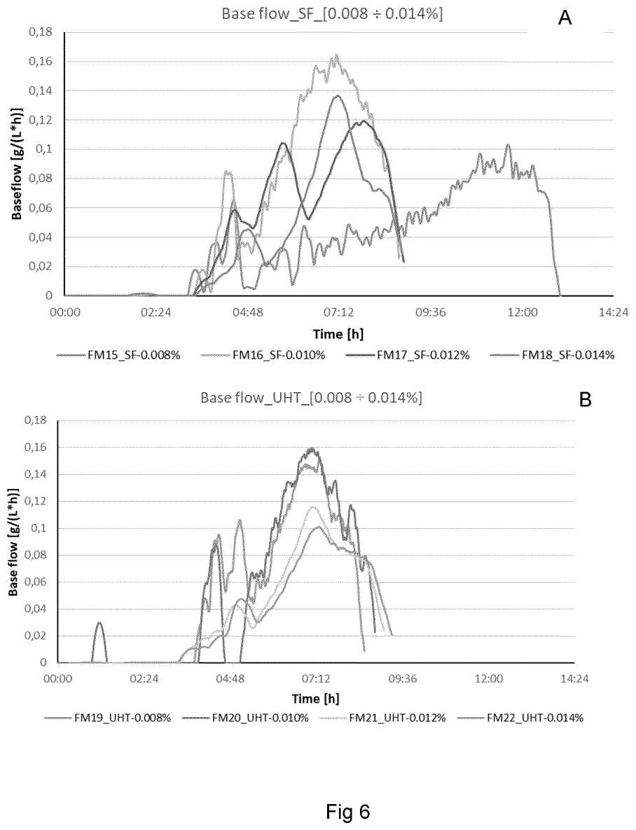

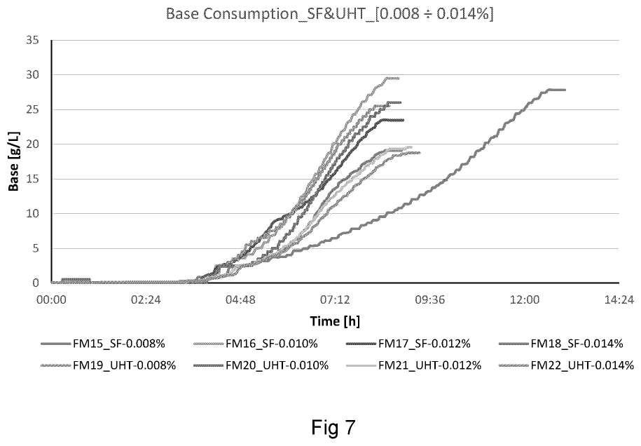

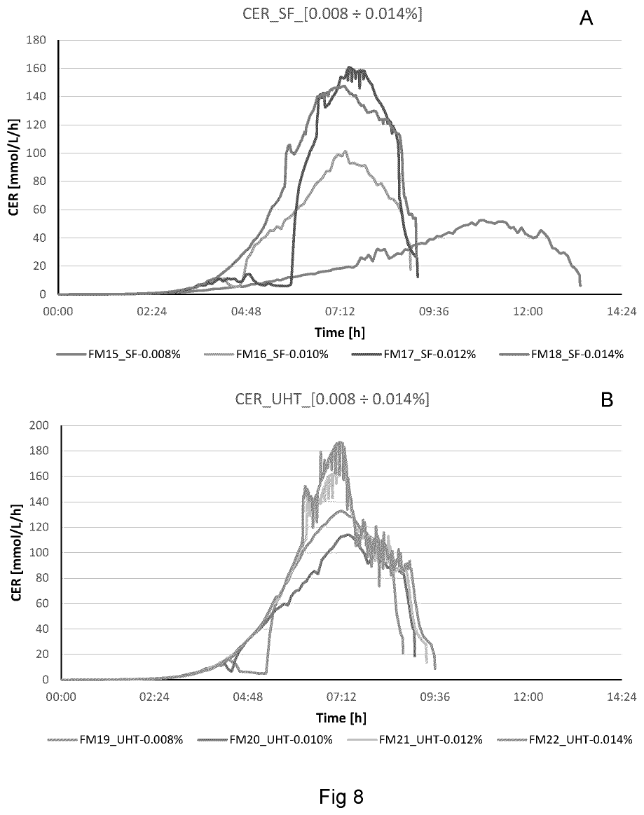

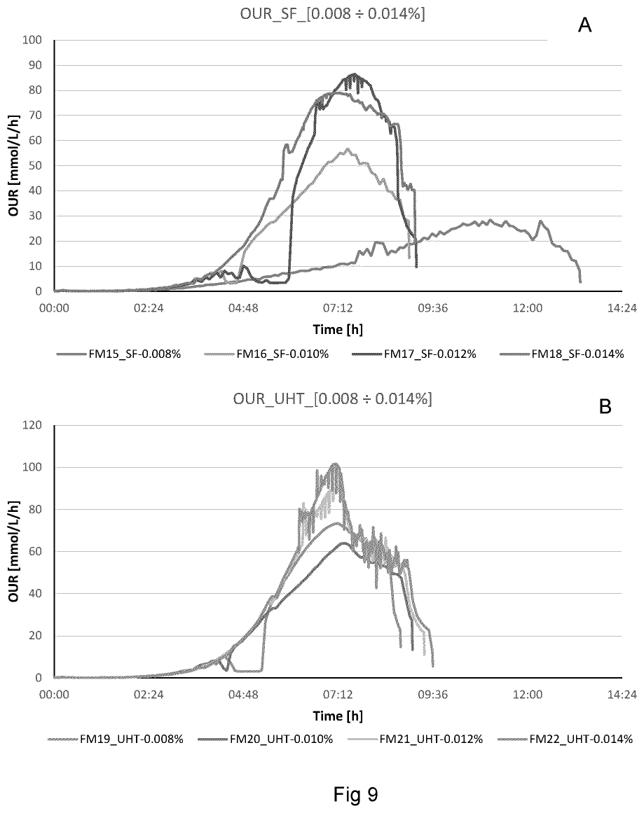

During the course of the cultivation the following online parameters were monitored and recorded: DO (FIG. 5), rate of base consumption (FIG. 6), total base consumption (FIG. 7), CER (Carbon dioxide Evolution Rate) and OUR (Oxygen Uptake Rate) (FIG. 8 and FIG. 9). These measurements were employed to evaluate if and to which extent respiratory metabolism was established during the cultivation process.

FIG. 5 shows Dissolved Oxygen (DO) profiles for aerated cell cultivations in standard medium supplemented with Heme from dry biomass powder either sterile filtered (SF, upper graph, A) or UHT treated (lower graph, B). The tested concentration levels of heme (i.e., 0.008%, 0.010%, 0.012% and 0.014%) were identical for both sterilization treatment.

FIG. 6 shows Rate of base consumption for aerated cell cultivations in standard medium supplemented with Heme from dry biomass powder either sterile filtered (SF, upper graph, A) or UHT treated (lower graph, B). The tested concentration levels of heme (i.e., 0.008%, 0.010%, 0.012% and 0.014%) were identical for both sterilization treatment.

FIG. 7 shows total base consumption for aerated cell cultivations in standard medium supplemented with Heme from dry biomass powder either sterile filtered (SF) or UHT treated. The tested concentration levels of heme (i.e., 0.008%, 0.010%, 0.012% and 0.014%) were identical for both sterilization treatment.

FIG. 8 shows Carbon dioxide Evolution Rate CER for aerated cell cultivations in standard medium supplemented with Heme from dry biomass powder either sterile filtered (SF, upper graph, A) or UHT treated (lower graph, B). The tested concentration levels of heme (i.e., 0.008%, 0.010%, 0.012% and 0.014%) were identical for both sterilization treatment.

FIG. 9 shows Oxygen Uptake Rate (OUR) for aerated cell cultivations in standard medium supplemented with Heme from dry biomass powder either sterile filtered (upper graph, A) or UHT treated (lower graph, B). The tested concentration levels of heme (i.e., 0.008%, 0.010%, 0.012% and 0.014%) were identical for both sterilization treatment.

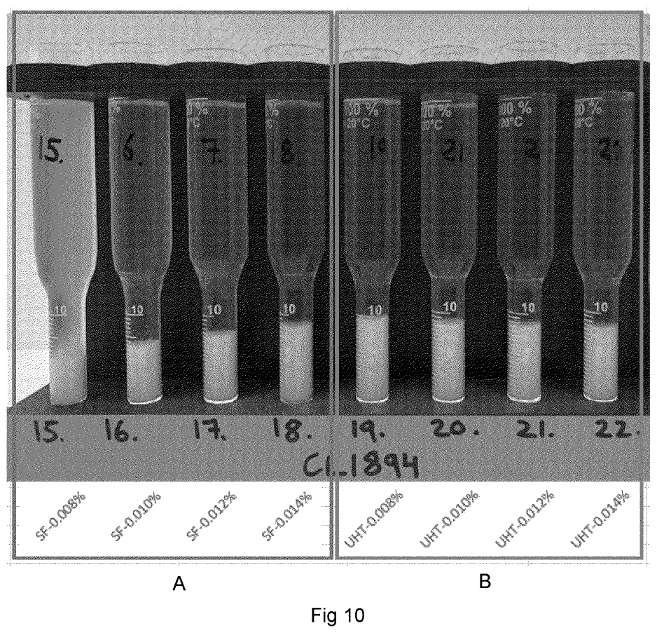

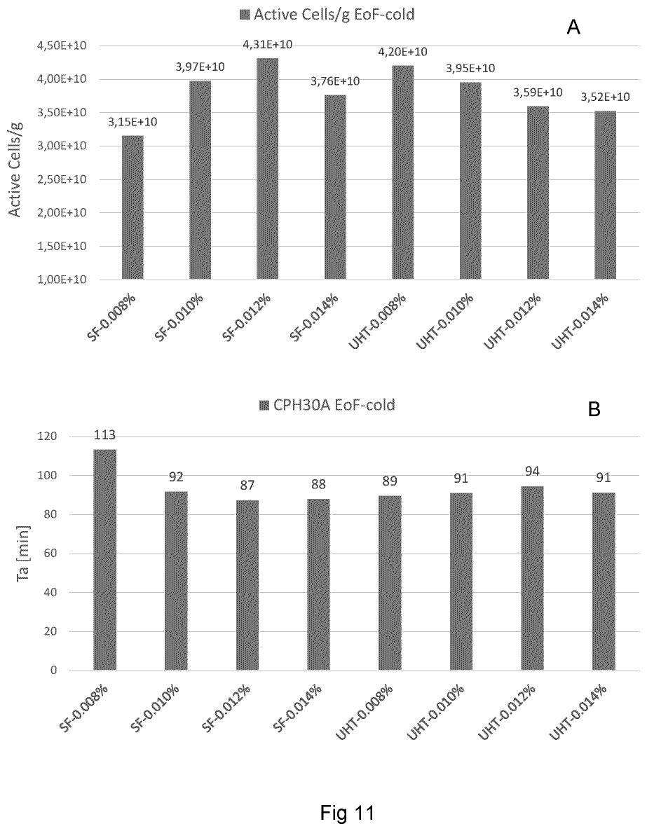

At the end of the cultivation the respiration performance of each aerobic culture was further assessed on the obtained final fermentate by off-line measurements of the PCV (Packed Cell Volume), active cell count (through flow cytometry) and acidification activity (0.1% inoculation rate across all samples) as shown in FIG. 10 and FIG. 11, respectively.

FIG. 10 shows Packed Cell Volume (PCV) % measurements for End of Fermentation (EoF) culture broth samples from aerated cell cultivations carried out with addition of Heme from dry biomass powder either sterile filtered (left hand side, A) or UHT treated (right hand side, B). The tested concentration levels of heme (i.e., 0.008%, 0.010%, 0.012% and 0.014%) were identical for both sterilization treatment.

FIG. 11 shows active cell counts (upper diagram, A) and acidification activities (lower diagram, B) measured for End of Fermentation (EoF) cold (i.e.: <10° C.) culture broth samples as a function of the supplemented heme levels (i.e., 0.008%, 0.010%, 0.012% and 0.014%) for both the first set of four aerobic cultivations carried out with sterile filtered (SF) heme solution and the second group of aerobic cultivations performed using UHT treated heme solutions.

Under aerated conditions and in the presence of heme the onset of respiratory metabolism occurred in the case of the sterile filtered heme-containing biomass powder starting from 0.010% while for the UHT treated powder aerobic respiration was observed for the full range of concentrations tested. This could indicate that 0.008% filter sterilized Heme biomass powder was not enough to sufficiently activate the bd-type cytochrome oxidase having therefore a negative impact on the respiratory capability of the cells.

Interestingly it was observed that larger PCV (%) values were obtained for increased concentrations of sterile filtered Heme biomass powder while in the case of UHT treated Heme biomass powder, PCV values were inversely related to the amount of heme-supplement that was added to the cultivation medium (FIG. 10).

Acidification activities were in line with the usual values obtained with reference (typically with a Ta around 88 min, data not shown) except for the case of the cell culture where the standard medium was supplemented with 0.008% filter sterilized Heme biomass powder (i.e., Ta equal to 113 min) as shown in the lower diagram of FIG. 11. This was in agreement with the fact that effective respiration did not occur in this last case.

The best result in 2 L fermenter trials according to this example was Fermentate-PCV>9.5% and Ta=89 min, which was observed for the Heme biomass powder obtained for the UHT treated material at a concentration of 0.008% (w/w) powder. This concentration was equivalent to 80 mg of heme powder per liter of cultivation medium, corresponding to 4-8 ppm of free heme. Translated into larger scale, this would mean use of 2.4 kg of Heme biomass powder for the production in a full scale 30 m³ aerated batch.

This example indicates that both sterile filtered and UHT treated stock solutions of dissolved biomass powder support respiration of the model Lactococcus strain.

For material provided by sterile filtration there is a dose-response effect showing increased cell volume for increasing concentration of the sterile filtered stock solution being added. A similar trend was observed for the number of active cells as measured by Flow Cytometry (data not shown).

For UHT treated material a dose-response effect is observed showing decreasing cell volumes for in-creasing concentration of the UHT treated stock solution. A similar decreasing trend was observed for the number of active cells as measured by Flow Cytometry (data not shown).

For the tested concentrations of dissolved dried biomass provided, all samples enabled acceptable acidification performance when tested in a standardized milk-based setup.

1: A method for obtaining a microbial culture, said method comprises the steps of:
(i) culturing at least one microbial strain in a culture medium under aeration and obtaining a fermentate,
(ii) harvesting from the fermentate said microbial strain to obtain the microbial culture, wherein the culture medium comprises non-protein-bound heme.

2: The method according to claim 1, wherein the non-protein-bound heme is microbially produced.

3: The method according to claim 1, wherein the non-protein-bound heme is produced by expression from the genus Aspergillus, Pichia, Bacillus, Saccharomyces, or Escherichia.

4: The method according to claim 1, wherein the non-protein-bound heme is sterilized by filtration or heat inactivation.

5: The method according to claim 1, wherein the non-protein-bound heme is added to a concentration of between about 0.1 g/kg fermentate and about 10 g/kg fermentate.

6: The method according to claim 1, said method further comprising:
(iii) concentrating the microbial culture to obtain a concentrated microbial culture.

7: The method according to claim 1, said method further comprising:
(iv) freezing or drying said microbial culture to obtain a frozen or dried microbial culture.

8: The method according to claim 7, said method further comprising:
(v) packing said frozen microbial culture or the dried microbial culture obtained in step (iv).

9: The method according to claim 1, wherein the culture medium does not comprise a non-vegetarian compliant non-protein-bound heme.

10: The method according to claim 1, wherein the microbial strain is Lactococcus lactis DSM 24648.

11: The method according to claim 1, wherein the culture medium further comprises a heat stabilizing compound selected from the group consisting of: polyols, sugars, biopolymers, amino acids, salt, polymers and non-ionic detergents.

12: A culture obtained by the method according to claim 1.

13: A culture or a culture medium comprising at least one non-protein-bound heme.

14: A method of preparing a food product, feed product, a pharmaceutical product, a dairy flavor and a cheese flavoring product,
said method comprising adding an effective amount of the culture according to claim 12 to a food, feed or pharmaceutical product starting material and keeping the inoculated culture under conditions where the at least one microbial strain is metabolically active.

15: A fermented food, feed or pharmaceutical product obtained by the method of claim 1.

16. (canceled)

17: A food product, feed product, a pharmaceutical product, a dairy flavor or a cheese flavoring product, comprising the culture according to claim 12.

18: A food product, feed product, a pharmaceutical product, a dairy flavor or a cheese flavoring product, comprising the culture according to claim 13.

19: A method of preparing a food product, feed product, a pharmaceutical product, a dairy flavor and a cheese flavoring product,
said method comprising adding an effective amount of the culture according to claim 13 to a food, feed or pharmaceutical product starting material and keeping the inoculated culture under conditions where the at least one microbial strain is metabolically active.

20: The culture or a culture medium according to claim 13, comprising the microbial strain Lactococcus lactis DSM 24648.

21: The culture or a culture medium according to claim 13, further comprises a heat stabilizing compound selected from the group consisting of: polyols, sugars, biopolymers, amino acids, salt, polymers and non-ionic detergents.
